(19) United States
(12) Patent Application Publication
(10) Pub. No.: US 2022/0020518 A1
(43) Pub. Date: Jan. 20, 2022

(54) APPARATUS AND METHOD FOR CONTROL OF AN ELECTROPERMANENT MAGNETIC SYSTEM

(71) Applicant: Weinberg Medical Physics, Inc., North Bethesda, MD (US)

(72) Inventor: Irving N. WEINBERG, North Bethesda, MD (US)

(21) Appl. No.: 17/378,268

(22) Filed: Jul. 16, 2021

Related U.S. Application Data
(60) Provisional application No. 63/053,121, filed on Jul. 17, 2020.

(51) Int. Cl. H01F 7/02; G01R 33/383

(52) U.S. Cl. CPC H01F 7/0294; G01R 33/383

(57) ABSTRACT

An apparatus and method are provided for implementing feedback control of the electropermanent magnets and also collecting information about magnetic fields emanating from a volume of interest containing a living being

### CROSS REFERENCE AND PRIORITY CLAIM

This application claims priority to U.S. Provisional Patent Application Ser. No. 63/053,121, entitled “APPARATUS AND METHOD FOR CONTROL OF AN ELECTROPERMANENT MAGNETIC SYSTEM” filed Jul. 17, 2020, the entirety of which is incorporated by reference.

### FIELD

Disclosed embodiments are directed, generally, to an apparatus and method for controlling an electropermanent magnet system.

It is known that the magnetic field generated by electromagnets can be controlled with feedback from magnetic sensors. Electropermanent magnets consist of coils that surround a core of permanent magnet material. The term permanent magnet material is defined as a material which becomes magnetized after immersion in an externally-applied magnetic field, and which retains some magnetization after that magnetic field is removed. After an electrical current is passed through the core, the electropermanent magnet retains its magnetization. It is possible to make a magnetic resonance imaging (MRI) system using electropermanent magnets. Such an MRI system achieves power savings as compared to resistive MRI systems, since the electrical current through the coil only needs to run for a short portion of the duty cycle to sustain a magnetic field adjacent to the electropermanent magnet. The same electropermanent magnets can be used to manipulate magnetic materials or to generate magnetic fields for stimulation of neurons or to collect magnetic particle images (MPI).

### SUMMARY

Disclosed embodiments describe an apparatus and method for implementing feedback control of the electropermanent magnets and also collecting information about magnetic fields emanating from a volume of interest containing a living being.

In some embodiments, the apparatus includes one or more modules, each module including at least one control sub-module, at least one electropermanent sub-module, and at least one monitoring sub-module. The at least one monitoring sub-module contains at least one measurement structure for measuring a magnetic field at a location of the at least one monitoring sub-module.

In some embodiments method of controlling a magnetic field generation comprises measuring a magnetic field using a spin status of at least one sample within at least one monitoring sub-module, and controlling the magnetic field generated by adjusting or maintaining the magnetic field based on the measured magnetic field compared to a predetermined desired magnetization state.

### BRIEF DESCRIPTION OF FIGURES

FIG. 1 illustrates an embodiment of an apparatus including a module containing a plurality of sub-modules; and

FIG. 2 is a flowchart of an exemplary imaging or interventional procedure according to the disclosed embodiment.

### DETAILED DESCRIPTION

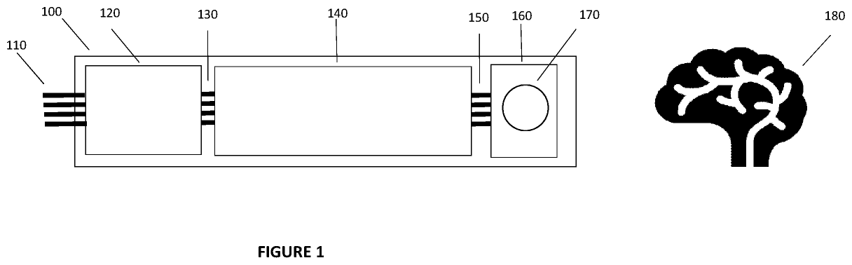

FIG. 1 shows an example of an embodiment of an apparatus. The apparatus consists of one or more electropermanent magnet modules 100 (subsequently referred to as “module” in this specification). Each module may be covered partially or completely with active or passive shielding materials (not shown) to reduce the influence of modules on one another. Each module has electrical and/or magnetic coupling contacts 110 that are used to supply power and/or instructions to the module from a computer and/or power supply (not shown). Within module 100 is a board and/or integrated circuit denoted as sub-module 120 that controls, powers and/or monitors the activities of the module. Within module 100 is an electropermanent magnet or electropermanent magnets denoted as sub-module 140 that generate a magnetic field when energized and/or controlled by circuit sub-module 120 via contacts 130. Within module 100 may be an electron spin resonance or other magnetometer denoted as monitoring sub-module module 160, which may contain a source of free electrons 170 whose electron spin resonance is measured (for example, by an antenna connected to an amplifier) within sub-module 160. Monitoring sub-module may be energized and/or controlled by control sub-module 120 via contacts 150. Monitoring sub-module 160 may also control electropermanent magnet sub-module 140 directly without the intervention of control sub-module 120. Within one meter of module 100 is a subject or sample 180, which is assayed, estimated, and/or affected by the magnetic field generated by module 100. The subject or sample is defined as being within a volume of interest to a user. The terms region of interest and volume of interest are used interchangeably in this specification.

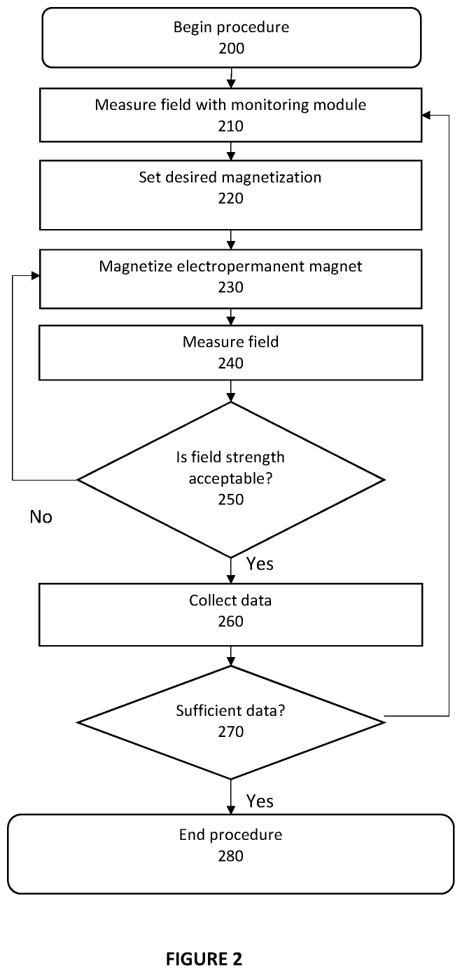

FIG. 2 shows an example of one embodiment of a method. An imaging or interventional procedure is initiated by a user or a computer 200. The procedure may be an MRI, MPI, or magnetic stimulation of one or more portions of an object in a region of interest. In step 210, the magnetic field near and/or in the module may then be measured with the monitoring sub-module using electron spin resonance or some other measurement tool. The desired magnetization state of the electropermanent magnet sub-module (or the upper and lower bounds of said desired state) 220 by the user or computer or the control sub-module or a combination of these. A magnetic field is generated by electropermanent module 230. The magnetic field near or in the module may again be measured with the monitoring sub-module using electron spin resonance or another measurement method 240. The control sub-module or other module component will then assess 250 whether the magnetic field strength monitored with the monitoring sub-module is acceptable using the spin state identified in the electron spin resonance (or using some other measurement of magnetic field). If the magnetic field strength is not acceptable, the magnetization of the electropermanent magnet will be adjusted and monitored again. It is understood that the term “magnetization of the electropermanent magnet” refers to the magnetization of the core material in the electropermanent magnet. It is understood that the term “acceptable” is defined as having the measured magnetic field strength be within the parameters set in step 220. It is understood that the magnetic field in the volume of interest may be extrapolated from the measured magnetic field. If the magnetic field strength is acceptable, data will be collected concerning the volume of interest 260, said collection for example being obtained with radiofrequency irradiation of and/or radiofrequency reception (using antennas or other means) with respect to a volume of interest to form an image of an object in the volume of interest. The user or computer or module or sub-module will assess 270 whether the data collected by the apparatus is sufficient to form an image or collect other data as needed to complete the procedure. If so, the procedure is completed 280. Otherwise additional recursions are obtained.

As discussed above in the description of the Figures, the apparatus of the invention consists of at least one module 100 within a meter of a volume of interest that contains an object of interest 180. Each module may have shielding to reduce the influence of the magnetic fields generated by one module on another. The shielding may be passive (for example, iron or mu-metal) or may be active (for example, a current-carrying coil or current-carrying sheet of conductive metal).

Instructions may be sent from a computer to each module via connectors 110, and power may also be sent from via connectors 110. Connections or connectors 110 may be implemented with wire, or via optical or wireless means. A control sub-module 120 controls operation of the module 100, said control including implementation of a feedback loop within the module 100 so that the electropermanent magnet sub-module 140 is generating an appropriate magnetic field as per the settings prescribed via connections 110.

It should be understood that a source or sources for generating current needed to actuate electropermanent magnet sub-module may be wholly or partially within the control sub-module 120 or may be wholly or partially with the electropermanent sub-module 140 or may be elsewhere within the module 100. Said source or sources may include one or more capacitors, switches, relays, or resistors to form an H-bridge or other circuit that compresses energy input to the module via connections 110 and 130 into a shorter and/or more powerful current through components within the electropermanent sub-module 140.

It should be understood that electro-permanent sub-module 140 may contain magnetizable core material (for example, AlNiCo rods) and coils or conductive sheets or other conductive or magnetizable materials for generating a magnetic field. Sub-module 140 may contain a magnetostrictive material to generate a magnetic field that depends on a voltage applied to the magnet. Said magnetic field may be used to magnetize the core material and/or to generate a magnetic field as needed to study object or objects 180 in the field of interest. Object 180 may be animate or inanimate and may be human or non-human.

It should be understood that the terms “field of interest” or “field of view” refer to and include regions containing object or objects 180 that are of interest for a user wishing to describe or alter the function and/or anatomy of said objects.

Monitoring sub-module 160 may contain at least one coil or other electrical antenna or electromagnetic cavity or other measurement structure as needed to assess the magnetic spin state of a sample 170 via electron spin resonance or other field measurement principles. In an embodiment, electron spin resonance is used to assess the state of a free-electron-containing sample 170 (for example, Templo material) in order to collect information about the sample 170., The purpose of electron spin resonance may be to use the properties of the state of the sample 170 in order to determine the strength of the magnetic field in the vicinity (that is within one meter) of the monitoring sub-module 170. The magnetic field information collected by the monitoring sub-module is shared with the other sub-modules in the module via connections 130 and/or 150 or via other connections that are not shown and may also be shared with a computer via connections 110. It should be understood that the information may be shared between other sub-modules via connections that are not shown in FIG. 1, for example via a connection from monitoring sub-module 160 to control sub-module 120.

In an alternative embodiment, the monitoring sub-module may utilize optical measurement of a sample within the monitoring sub-module to assess the magnetic field. For example, the sample may be a diamond with a nitrogen-vacancy center.

It should be understood that electron-spin resonance measurements can be very rapid, for example within less than a microsecond. This rapidity may be advantageous when setting the module to a desired magnetic field quickly. It should be understood that the frequencies for electron spin-resonance with presently available wi-fi technologies (e.g. 1-10 GHz) may be a good fit for the magnetic field that can be generated with electropermanent magnets (e.g. 1-100 mT).

It should be understood that the term “sub-module” is a term that refers to the presence of specified functionality and not necessarily a physical location. Consistent with that meaning, a sub-module need not be in a different physical location than another sub-module. For example, the control sub-module 120 may be integrated physically within the electropermanent sub-module 140 and/or within the monitoring sub-module 160.

It should be understood that the term “module” is used to describe functionality and not necessarily physical location. Consistent with that meaning, for example a monitoring sub-module may be in a different physical housing than the control sub-module or the electropermanent sub-module, and still be considered as a single apparatus as taught by this disclosure.

The magnetic fields measured by monitoring sub-module 160 can be used to collect information about the magnetic fields emanated by sample 180. For example, It should be understood that the magnetic field within a region or volume of interest can be assessed via measurement at the border of the region (for example via Gauss' law of electromagnetism, or an approximation to said law). If sample 180 is a human's or non-human animal's brain, said information about the magnetic field at sample 180 may be used to implement magnetoencephalography. If sample 180 is a human brain, said information about the magnetic field at sample 180 may be used to alter the magnetic field generated by the electropermanent magnet sub-module to implement transcranial magnetic stimulation. A device containing one or more modules 100 may therefore be used to perform multiple tasks within moving sample 180. Such tasks may include magnetoencephalography, magnetic resonance imaging, magnetic particle imaging, and transcranial magnetic stimulation. It should be understood that the terms “brain” and “magnetoencephalogram” are general terms and are intended to also to represent objects and activities relating to other neuronal or nervous tissues. For example, the apparatus and/or method may be used to collect data about pain stimuli perceived in a peripheral nerve or nerve root and/or to relieve pain in a peripheral nerve or nerve root.

Moreover, those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

1. An apparatus for controlling an electropermanent magnetic field, the apparatus comprising:
at least one control sub-module,
at least one electropermanent sub-module,
and at least one monitoring sub-module,
wherein the at least one monitoring sub-module contains at least one measurement structure for measuring a magnetic field at a location of the at least one monitoring sub-module.

2. The apparatus as in claim 1, where the measurement structure contains a sample of free electrons that are excited via electron spin resonance.

3. The apparatus of claim 2, wherein the at least one monitoring sub-module further comprises an antenna for measuring the magnetic spin state of the sample of free electrons.

4. The apparatus of claim 1, wherein the at least one electropermanent sub-module generates the magnetic field measured by the measurement structure.

5. The apparatus of claim 1, wherein the at least one control sub-module controls the generation of the magnetic field by the at least one electropermanent sub-module.

6. The apparatus of clam 1, wherein the at least one monitoring sub-module controls adjustment of the magnetic field generated by the at least one electropermanent magnet sub-module based on the measured magnetic field.

7. The apparatus of claim 1, wherein the at least one control sub-module, the least one electropermanent sub-module, and the at least one monitoring sub-module form a feedback loop so that the magnetic field generated by the electropermanent magnet sub-module is consistent with settings prescribed to the at least one control sub-module.

8. The apparatus of claim 1, wherein the spin status of the sample in the measurement structure is used to collect magnetic resonance images of an object.

9. The apparatus of claim 1, wherein the spin status of the sample in the measurement structure is used to collect magnetic encephalograms of an object.

10. The apparatus of claim 1, wherein the spin status of the sample in the measurement structure is used to alter the magnetic field of an object in a region of interest.

11. The apparatus of claim 1, wherein data obtained with the measurement structure is used to alter the magnetic field of an object in a region of interest.

12. A method of controlling a magnetic field generation, the method comprising:
measuring a magnetic field using a spin status of at least one sample within at least one monitoring sub-module, and
controlling the magnetic field generated by adjusting or maintaining the magnetic field based on the measured magnetic field compared to a predetermined desired magnetization state.

13. The method of claim 12, wherein the spin status of the sample in a measurement structure is assessed with electron spin resonance.

14. The method of claim 12, wherein the spin status of the sample in a measurement structure is used to collect magnetic resonance images of an object in a region of interest.

15. The method of claim 12, wherein the spin status of the sample in a measurement structure is used to collect magnetic encephalograms of an object in a region of interest.

16. The method of claim 12, wherein data obtained from the measurement structure is used to collect magnetic resonance images of an object in a region of interest.

17. The method of claim 12, wherein data obtained from the measurement structure is used to collect magnetic encephalograms of an object in a region of interest.

18. The method of claim 12, wherein data obtained from the measurement structure is used to estimate and/or affect the magnetic field of an object in a region of interest.

19. The method of claim 12, wherein the magnetic field is generated by one or more electropermanent magnets.

20. The method of claim 12, wherein the magnetic field is adjusted and measured again to determine if an adjusted magnetic field strength is equal to a predetermined magnetic field strength.

21. The method of claim 20, wherein imaging of an object in a region of interest is commenced in response to the adjusted magnetic field strength being equal to the predetermined magnetic field strength.
